(19) United States
(12) Patent Application Publication
(10) Pub. No.: US 2020/0253585 A1
(43) Pub. Date: Aug. 13, 2020

(54) METHODS AND APPARATUSES FOR COLLECTING ULTRASOUND IMAGES DEPICTING NEEDLES

(71) Applicants: Abraham Neben, Guilford, CT (US); Brenda Gonyeau, Feeding Hills, MA (US)

(72) Inventors: Abraham Neben, Guilford, CT (US); Brenda Gonyeau, Feeding Hills, MA (US)

(73) Assignee: Butterfly Network, Inc., Guilford, CT (US)

(21) Appl. No.: 16/789,149

(22) Filed: Feb. 12, 2020

Related U.S. Application Data
(60) Provisional application No. 62/805,293, filed on Feb. 13, 2019.

(51) Int. Cl. A61B 8/00; A61B 8/08

(52) U.S. Cl. CPC A61B 8/4494; A61B 8/0841; A61B 8/465

(57) ABSTRACT

Aspects of the technology described herein relate to collection of ultrasound images depicting needles. Some embodiments include an ultrasound device having a transducer array and configured to use an aperture having a centroid that is closer to a first long side of the transducer array than a second long side of the transducer array. Certain embodiments include instructing a user to begin to insert a needle adjacent at a location that is to a particular long side of the transducer array. Some embodiments include receiving a selection of a particular long side of the transducer array. Certain embodiments include determining that a user has begun to insert a needle at a location that is adjacent to a particular long side of the transducer array.

### CROSS-REFERENCE TO RELATED APPLICATIONS

This application claims the benefit under 35 U.S.C. § 119(e) of U.S. Patent Application Ser. No. 62/805,293, filed Feb. 13, 2019 under Attorney Docket No. B1348.70129US00, and entitled “METHODS AND APPARATUSES FOR COLLECTING ULTRASOUND IMAGES DEPICTING NEEDLES,” which is hereby incorporated herein by reference in its entirety.

### FIELD

Generally, the aspects of the technology described herein relate to collection of ultrasound data. Some aspects relate to methods and apparatuses for collecting ultrasound images depicting needles.

### BACKGROUND

Ultrasound probes may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect of the present application, an apparatus includes an ultrasound device having a transducer array, the ultrasound device configured to use an aperture having a centroid that is closer to a first long side of the transducer array than a second long side of the transducer array.

In some embodiments, the aperture is rectangular. In some embodiments, the aperture is between or equal to approximately 1-10 mm along an elevational dimension of the transducer array. In some embodiments, the aperture covers between or equal to approximately 20-30% of an elevational dimension of the transducer array. In some embodiments, the aperture is not centered along an elevational dimension of the transducer array. In some embodiments, the aperture is directly adjacent to the first long side of the transducer array. In some embodiments, the aperture is centered with respect to an azimuthal dimension of the transducer array. In some embodiments, the aperture extends along an entire length of an azimuthal dimension of the transducer array. In some embodiments, the aperture does not extend along an entire length of an azimuthal dimension of the transducer array. In some embodiments, the aperture is not centered with respect to an azimuthal dimension of the transducer array. In some embodiments, the aperture is directly adjacent to a first short side of the transducer array. In some embodiments, the aperture is not directly adjacent to the first long side of the transducer array. In some embodiments, the aperture is not rectangular. In some embodiments, a width of the aperture along an elevational dimension of the transducer array varies with imaging depth. In some embodiments, the ultrasound device is further configured to average, with a weighted sum along an azimuthal dimension of the transducer array, signals received by the aperture.

According to another aspect of the present application, an apparatus includes a processing device in operative communication with an ultrasound device having a transducer array, the processing device configured to instruct a user to begin to insert an object into a patient at a location that is adjacent to a particular long side of the transducer array.

In some embodiments, the processing device is configured, when instructing the user to begin to insert the object into the patient at the location that is adjacent to the particular long side of the transducer array, to instruct the user to insert the object into the patient at a location that is adjacent to a particular face of the ultrasound device. In some embodiments, the particular face of the ultrasound device is adjacent to the particular long side of the transducer array. In some embodiments, the processing device is configured, when instructing the user to insert the object into the patient at the location that is adjacent to the particular face of the ultrasound device, to instruct the user to insert the object into the patient at a location that is adjacent to a particular face of the ultrasound device having a particular marking. In some embodiments, the processing device is configured, when instructing the user to insert the object into the patient at the location that is adjacent to the particular face of the ultrasound device, to instruct the user to insert the object into the patient at a location that is adjacent to a particular face of the ultrasound device lacking a particular marking. In some embodiments, the processing device is further configured to configure the ultrasound device to use an aperture having a centroid that is closer to the particular long side of the transducer array than another long side of the transducer array. In some embodiments, the object is a needle.

According to another aspect of the present application, an apparatus includes a processing device in operative communication with an ultrasound device having a transducer array, the processing device configured to receive a selection of a particular long side of the transducer array.

In some embodiments, the processing device is further configured to display two options, each corresponding to a particular long side of the transducer array, and the processing device is configured, when receiving the selection of the particular long side of the transducer array, to receive a selection of one of the two options. In some embodiments, the processing device is further configured to configure the ultrasound device to use an aperture having a centroid that is closer to the particular long side of the transducer array than another long side of the transducer array. In some embodiments, the processing device is further configured to display an indication of the particular long side of the transducer array that is closer to the aperture by highlighting one of the two options that corresponds to the particular long side of the transducer array. In some embodiments, the processing device is further configured to display an option, and the processing device is configured, when receiving the selection of the particular long side of the transducer array, to receive a selection of the option. In some embodiments, the processing device is configured, based on receiving the selection of the option, to configure the ultrasound device to switch from using an aperture having a centroid that is closer to a first long side of the transducer array to using an aperture having a centroid that is closer to a second long side of the transducer array.

Some aspects include a method of performing the actions that the apparatuses of the above aspects and embodiments are configured to perform. Some aspects include at least one non-transitory computer-readable storage medium storing processor-executable instructions that, when executed by at least one processor, cause the at least one processor to perform the actions that the apparatuses of the above aspects and embodiments are configured to perform

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear

FIGS. 1 and 2 illustrate schematic diagrams of an example of a needle being inserted into a subject and imaged with a transducer array, in accordance with certain embodiments described herein;

FIG. 3 schematically illustrates an example aperture on a transducer array, in accordance with certain embodiments described herein;

FIG. 4 schematically illustrates an example aperture on a transducer array, in accordance with certain embodiments described herein;

FIG. 5 schematically illustrates an example aperture on a transducer array, in accordance with certain embodiments described herein;

FIG. 6 schematically illustrates an example aperture on a transducer array, in accordance with certain embodiments described herein;

FIG. 7 schematically illustrates an example aperture on a transducer array, in accordance with certain embodiments described herein;

FIG. 8 schematically illustrates an example aperture on a transducer array, in accordance with certain embodiments described herein;

FIG. 9 schematically illustrates an example aperture on a transducer array, in accordance with certain embodiments described herein;

FIG. 10 schematically illustrates an example aperture on a transducer array, in accordance with certain embodiments described herein;

FIG. 11 schematically illustrates an example aperture on a transducer array, in accordance with certain embodiments described herein;

FIGS. 12-14 schematically illustrate examples of apertures that vary as a function of imaging depth;

FIGS. 15-17 schematically illustrate more examples of apertures that vary as a function of imaging depth;

FIG. 18 schematically illustrates an example aperture on a transducer array, in accordance with certain embodiments described herein;

FIG. 19 illustrates an example process in accordance with certain embodiments described herein;

FIG. 20 illustrates an example process in accordance with certain embodiments described herein;

FIG. 21 illustrates an example process in accordance with certain embodiments described herein;

FIG. 22 illustrates an example process in accordance with certain embodiments described herein;

FIG. 23 illustrates an example process in accordance with certain embodiments described herein;

FIG. 24 illustrates an example graphical user interface (GUI), in accordance with certain embodiments described herein;

FIG. 25 illustrates an example GUI, in accordance with certain embodiments described herein;

FIG. 26 illustrates an example GUI, in accordance with certain embodiments described herein;

FIG. 27 illustrates an example GUI, in accordance with certain embodiments described herein;

FIG. 28 illustrates an example GUI, in accordance with certain embodiments described herein;

FIG. 29 illustrates a view of an ultrasound device, in accordance with certain embodiments described herein;

FIG. 30 illustrates another view of the ultrasound device of FIG. 29, in accordance with certain embodiments described herein; and

FIG. 31 illustrates a schematic block diagram of an example ultrasound system upon which various aspects of the technology described herein may be practiced.

### DETAILED DESCRIPTION

An ultrasound transducer array generally produces an image by transmitting an ultrasound beam along a slice of tissue and collecting reflected ultrasound waves. The width of the slice is along the long axis of the transducer array and the depth of the slice is perpendicular to the face of the transducer array. An aperture may be a subset of the transducers of the transducer array used for transmitting and/or receiving one or more ultrasound beams for forming an ultrasound image. In some embodiments, the aperture may be the entire subset of the transducer array used for transmitting and/or receiving one or more ultrasound beams for forming an ultrasound image. The size of the aperture along the long axis of the transducer array may be referred to as the azimuthal aperture and the size of the aperture along the short axis of the transducer array may be referred to as the elevational aperture.

Certain medical procedures (e.g., biopsies and aspirations) may include insertion of a needle into a patient. Ultrasound imaging of the patient may be performed during insertion of the needle so the medical professional inserting the needle may monitor the needle and reduce the chance of accidentally injuring nerves, vessels, and other anatomical structures during the insertion of the needle. Ultrasound imaging of a needle in tissue may be performed with the needle either parallel to the slice (in-plane) or perpendicular to the slice (out-of-plane). An in-plane needle appears as a bright line. An out-of-plane needle appears as a bright point. This description relates to out-of-plane imaging.

For out-of-plane imaging of a needle, a user places an ultrasound device in contact with the patient being imaged, and begins to insert a needle into the patient at a location that is adjacent to the long side of the transducer array of the ultrasound device. In other words, the user inserts the needle into the patient in a direction that is approximately along the elevational dimension of the transducer array. (As referred to herein, inserting the needle into the patient “along” the elevational dimension of the transducer array may mean that, if the needle were projected into the plane of the transducer array, then during insertion the needle would move parallel to the elevational dimension of the transducer array.) An ultrasound probe may continuously generate ultrasound images by transmitting ultrasound beams through a slice of tissue and collecting reflecting ultrasound waves. The needle may become visible in the ultrasound images when it intersects the slice.

Ultrasound images generally depict anatomical structure averaged over a slice with a non-zero thickness. When a transducer array transmits and receives an ultrasound beam through a slice of tissue, the slice has a non-zero thickness. A smaller slice thickness may result in less smearing of structures in the elevational dimension, resulting in finer detail resolution, and thus, better image quality. The thickness of the slice may depend on the elevational aperture (for both transmitting and receiving) and focal depth, which may be tuned as a function of imaging depth. Near the focal depth, a larger elevational aperture generally may result in a narrower slice thickness. However, at superficial depths that are closer to the face of the transducer array, the slice thickness may be approximately equal to the elevational aperture. As described above, a smaller slice thickness may result in better image quality, and thus for applications that involve imaging superficial depths, such as detecting the location of needles inserted under the skin of a patient for example, smaller elevational apertures may typically be used.

Recently, ultrasound probes having large transducer arrays (with a large number of transducers along both the elevational and azimuthal dimensions) have been developed. These probes allow for flexibly optimizing apertures based on the anatomy being used. For further description of such ultrasound probes, see U.S. patent application Ser. No. 15/626,711 titled “UNIVERSAL ULTRASOUND IMAGING DEVICE AND RELATED APPARATUS AND METHODS,” filed on Jun. 19, 2017 and published as U.S. Pat. App. Publication No. 2017-0360399 A1 (and assigned to the assignee of the instant application), which is incorporated by reference herein in its entirety.

When a small elevational aperture is used in a large transducer array (in other words, small relative to the full elevational size of the transducer array), the aperture is typically centered in the elevational dimension of the transducer array. Thus, portions of the transducer array at the outer regions of the elevational dimension may not be used. This means that for out-of-plane needle imaging, in which the needle is inserted into the patient in a direction approximately along the elevational dimension of the transducer array when the array is placed in contact with the patient, the needle may need to first be inserted in a location of the patient that is adjacent to unused outer regions of the elevational dimension of the transducer array before the needle reaches a point that is adjacent to the aperture and intersects the imaging slice. Prior to this point in time, a user may be inserting the needle “blind,” meaning that the user cannot see the needle in ultrasound images collected by the probe. Blind needle insertion may increase the likelihood of accidentally injuring nerves, vessels, and other anatomical structures during the insertion of the needle. It may be undesirable to shift the ultrasound device such that the center of the transducer array (where the aperture is located) is adjacent to the insertion location of the needle on the subject, as portions of the transducer array may not be in contact with the subject, and this may introduce artifacts into ultrasound data collected by the ultrasound device.

The inventors have realized that when using a large transducer array, it may be helpful to use an aperture which is adjacent to the long side of the transducer array, rather than being centered with respect to the transducer array. If the needle is inserted adjacent to this long side of the transducer array, then the needle may be within the field of view of the aperture and therefore be visible on ultrasound images earlier in the insertion process than if the aperture were centered on the transducer array. Thus, the user may be blind for a shorter portion of the total insertion distance of the needle.

Some embodiments include an ultrasound device having a transducer array and configured to use an aperture that is closer to a particular long side of the transducer array than to the other long side of the transducer array. In some embodiments, a processing device in operative communication with the ultrasound device may configure the ultrasound device to use an aperture that is closer to a particular long side of the transducer array than to the other long side of the transducer array. In some embodiments, the processing device may provide an instruction to the user to begin to insert a needle into a patient at a location that is adjacent to the long side of the transducer array that is closer to the aperture. In some embodiments, the user may select a particular long side of the transducer array, and the processing device may configure the ultrasound device to use an aperture that is closer to the selected long side of the transducer array than to the other long side of the transducer array. In some embodiments, the processing device may determine (e.g., based on ultrasound images) that the needle is being inserted into a patient at a location that is adjacent to a particular long side of the transducer array, and configure the ultrasound device to use an aperture that is closer to this long side of the transducer array than to the other long side of the transducer array. In some embodiments, the processing device may collect ultrasound images using two apertures, one aperture closer to one long side of the transducer array and another aperture closer to the other long side of the transducer array. In such embodiments, the user may select one of the ultrasound images (i.e., one depicting a needle with acceptable brightness), and the processing device may configure the ultrasound device to use the aperture with which the selected ultrasound image was collected. Alternatively, in such embodiments, the processing device may determine which aperture to use going forward based on the ultrasound images. In some embodiments, the processing device may configure the ultrasound device collect an ultrasound image using an aperture covering the whole transducer array, determine based on the ultrasound image that the needle is being adjacent to a particular long side of the transducer array, and configure the ultrasound device to use an aperture that is adjacent to that particular long side of the transducer array.

It should be appreciated that while this description focuses on ultrasound imaging of needles, the same description may apply to other objects that are inserted into patients for medical procedures, such as hookwires and T-bars.

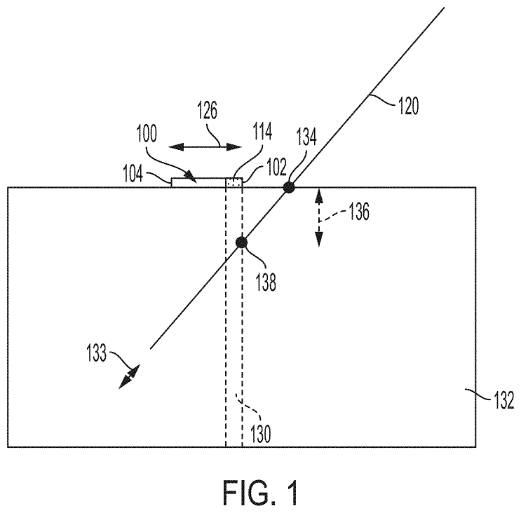

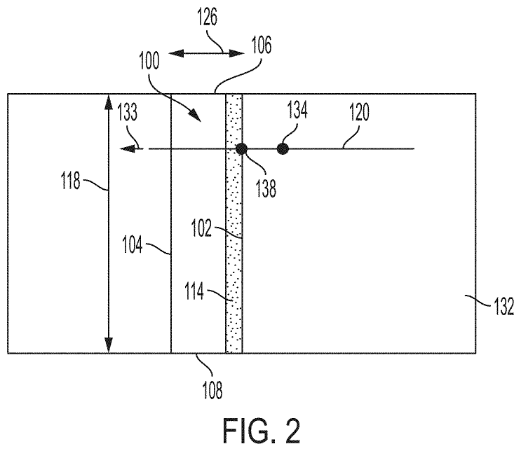

FIGS. 1-2 illustrate schematic diagrams of an example of a needle 120 being inserted into a subject 132 and imaged using an aperture 114 of a transducer array 100, in accordance with certain embodiments described herein. FIG. 1 is a side view of the transducer array 100 and FIG. 2 is a top view of the transducer array 100. The transducer array 100 is rectangular and has a first long side 102, a second long side 104, a first short side 106, a second short side 108, an elevational dimension 126, and an azimuthal dimension 118. The first long side 102 and the second long side 104 of the transducer array 100 are along the azimuthal dimension 118 of the transducer array 100. The first short side 106 and the second short side 108 are along the elevational dimension 126 of the transducer array 100.

The transducer array 100 includes a plurality of individual transducers arranged in an array, and the aperture 114 is a subset of the transducers of the transducer array 100 used for a particular function. For example, the aperture 114 may be the subset of the transducers of the transducer array 100 used for transmitting ultrasound waves and/or receiving ultrasound waves. The aperture 114 is rectangular and is narrow along the elevational dimension 126 of the transducer array 100 (relative to the elevational size of the transducer array 100). In some embodiments, the aperture 114 may be between or equal to approximately 1-10 mm along the elevational dimension 126 of the transducer array 100. In some embodiments, the aperture 114 may cover between or equal to approximately 20-30% of the elevational dimension 126 of the transducer array 100. The aperture 114 is not centered along the elevational dimension 126 of the transducer array 100. In particular, the aperture 114 is directly adjacent to the first long side 102 of the transducer array 100.

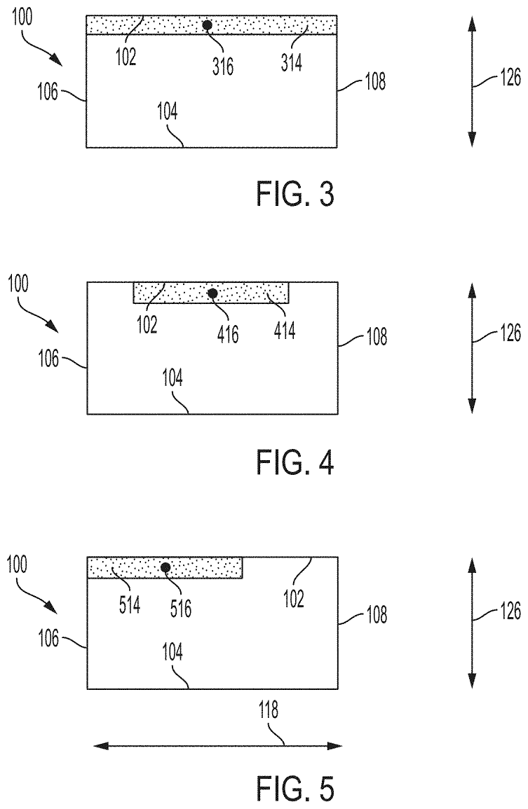

The aperture 114 transmits an ultrasound beam through a slice 130 of the subject 132. While in reality, the slice 130 may have non-uniform thickness, in FIG. 1 the slice 130 is illustrated with uniform thickness for simplicity. The needle 120 enters the subject 132 at the insertion point 134. (While the insertion point 134 is illustrated a distance away from the first long side 102 for clarity, in reality it may be helpful for the insertion point 134 to be as close as possible to the first long side 102.) The insertion point 134 is adjacent to the first long side 102 of the transducer array 100. The needle is inserted in a direction 133. The needle 120 is inserted along the elevational dimension 126 of the transducer array 100; as can be seen in the top view of FIG. 2, if the needle were projected into the plane of the transducer array 100, the direction 133 of insertion of the needle 120 would be parallel to the elevational dimension 126 of the transducer array. The needle 120 intersects the slice 130 at the intersection point 138. The intersection occurs when the needle 120 has been inserted a depth 136 into the subject 132. At this point, the needle 120 may become visible on ultrasound images. The user may therefore be “blind” with respect to the precise location of the needle 120 during insertion of the needle 120 prior to the time it intersects the slice 130, and thus throughout the insertion process to the depth 136. It should be noted that if the aperture 114 were to be centered along the elevational dimension 126 of the transducer array 100, the intersection between the needle 120 and the slice 130 would not occur until the needle 120 has been inserted to a greater depth into the subject 132 than with respect to the depth 136 illustrated in FIGS. 1-2, meaning that the user would be blind for a longer portion of the total insertion distance of the needle 120 into the subject 132. Thus, using the aperture 114 results in a shorter insertion distance during which the user is “blind” compared to using an aperture centered on the transducer array 100. As a result, using an offset aperture, such as aperture 114, may increase insertion accuracy and patient safety. It should be appreciated that this description applies equally to any of the apertures described herein, such as the apertures illustrated in FIGS. 3-18, or any of the variations or alternatives described.

FIG. 3 schematically illustrates an example aperture 314 on the transducer array 100, in accordance with certain embodiments described herein. The aperture 314 is rectangular and has a centroid 316. The aperture 314 is rectangular and is narrow along the elevational dimension 126 of the transducer array 100 (relative to the elevational size of the transducer array 100). The centroid 316 of the aperture 314 is closer to the first long side 102 of the transducer array 100 than the second long side 104 of the transducer array. In other words, the aperture 314 is not centered along the elevational dimension of the transducer array. The aperture 314 is directly adjacent to the first long side 102 of the transducer array 100, is centered with respect to the azimuthal dimension 118 of the transducer array 100, and extends along the entire length of the azimuthal dimension 118 of the transducer array 100. (The aperture 314 may be directly adjacent to the first long side 102 of the transducer array 100 when the aperture 314 includes the row of transducers making up the edge of the transducer array 100 adjacent to the first long side 102 of the transducer array 100.)

FIG. 4 schematically illustrates another example aperture 414 on the transducer array 100, in accordance with certain embodiments described herein. The aperture 414 is rectangular and has a centroid 416. The aperture 414 differs from the aperture 314 in that the aperture 414 does not extend along the entire length of the azimuthal dimension 118 of the transducer array 100. The aperture 414 is centered with respect to the azimuthal dimension 118 of the transducer array 100.

FIG. 5 schematically illustrates another example aperture 514 on the transducer array 100, in accordance with certain embodiments described herein. The aperture 514 is rectangular and has a centroid 516. The aperture 514 differs from the aperture 414 in that the aperture 514 is not centered with respect to the azimuthal dimension 118 of the transducer array 100. The aperture 514 is directly adjacent to the first short side 106 of the transducer array 100.

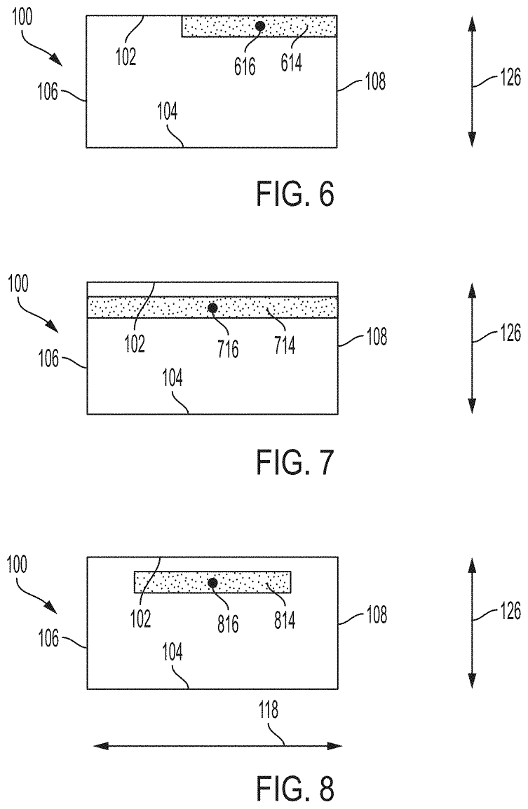

FIG. 6 schematically illustrates another example aperture 614 on the transducer array 100, in accordance with certain embodiments described herein. The aperture 614 is rectangular and has a centroid 616. The aperture 614 differs from the aperture 514 in that the aperture 614 is directly adjacent to the second short side 108 of the transducer array 100.

FIG. 7 schematically illustrates another example aperture 714 on the transducer array 100, in accordance with certain embodiments described herein. The aperture 714 is rectangular and has a centroid 716. The aperture 714 differs from the aperture 314 in that the aperture 714 is not directly adjacent to the first long side 102 of the transducer array 100. (The aperture 714 may not be directly adjacent to the first long side 102 of the transducer array 100 when the aperture 714 does not include the row of transducers making up the edge of the transducer array 100 adjacent to the first long side 102 of the transducer array 100.)

FIG. 8 schematically illustrates another example aperture 814 on the transducer array 100, in accordance with certain embodiments described herein. The aperture 814 is rectangular and has a centroid 816. The aperture 814 differs from the aperture 714 in that the aperture 814 does not extend along the entire length of the azimuthal dimension 118 of the transducer array 100. The aperture 814 is centered with respect to the azimuthal dimension 118 of the transducer array 100.

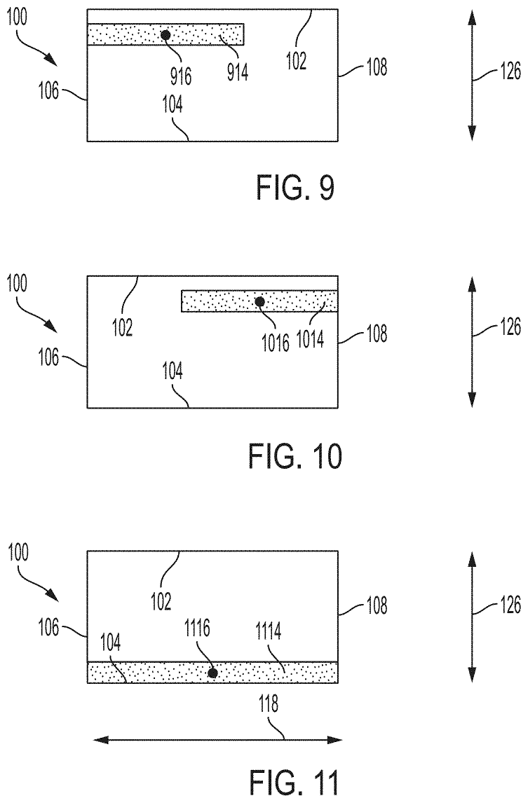

FIG. 9 schematically illustrates another example aperture 914 on the transducer array 100, in accordance with certain embodiments described herein. The aperture 914 is rectangular and has a centroid 916. The aperture 914 differs from the aperture 814 in that the aperture 914 is not centered with respect to the azimuthal dimension 118 of the transducer array 100. The aperture 914 is directly adjacent to the first short side 106 of the transducer array 100.

FIG. 10 schematically illustrates another example aperture 1014 on the transducer array 100, in accordance with certain embodiments described herein. The aperture 1014 is rectangular and has a centroid 1016. The aperture 1014 differs from the aperture 914 in that the aperture 1014 is directly adjacent to the second short side 108 of the transducer array 100.

FIG. 11 schematically illustrates an example aperture 1114 on the transducer array 100, in accordance with certain embodiments described herein. The aperture 1114 is rectangular and has a centroid 1116. The aperture 1114 differs from the aperture 314 in that the aperture 1114 is directly adjacent to the second long side 104 of the transducer array 100.

It should be appreciated that the apertures illustrated in FIGS. 3-11 are not limiting and other apertures may be used. For example, apertures that are the same as the apertures in FIGS. 4-10 but closer to the second long side 104 of the transducer array 100, rather than the first long side 102 of the transducer array 100, may be used. As another example, any non-rectangular aperture having a centroid that is closer to one long side of the transducer array 100 than another may be used. As another example, any aperture in which the majority of the ultrasound elements are closer to one long side of the transducer array 100 than another may be used. As another example, any aperture in which, if one finds the centroid of each column of the aperture that extends along the elevational direction of the aperture, the majority of the centroids are closer to one long side of the transducer array than another, may be used. As another example, any aperture which is not centered with respect to the elevational dimension of the transducer array may be used.

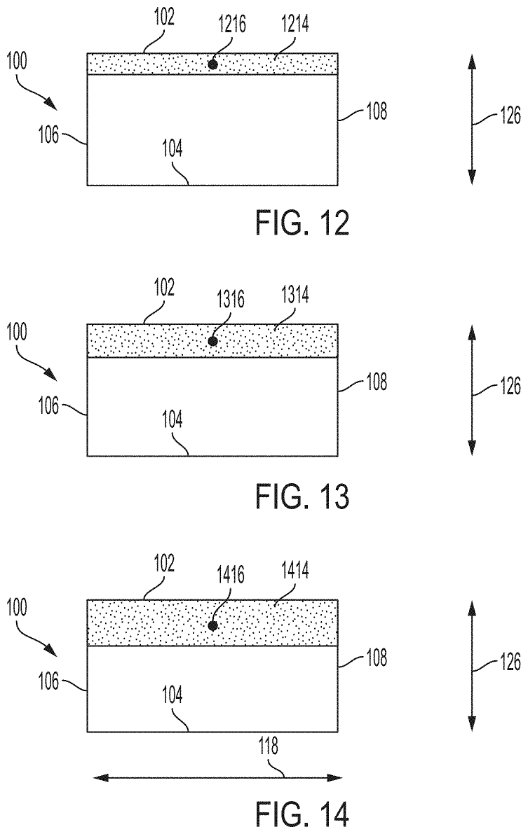

FIGS. 12-14 schematically illustrate examples of apertures that vary as a function of imaging depth, in accordance with certain embodiments described herein. FIG. 12 illustrates an aperture 1214 used for a first imaging depth, FIG. 13 illustrates an aperture 1314 used for a second imaging depth that is deeper than the first imaging depth, and FIG. 14 illustrates an aperture 1414 used for a third imaging depth that is deeper than the second imaging depth. The aperture 1414 is wider along the elevational dimension 126 than the aperture 1314, and the aperture 1314 is wider along the elevational dimension 126 than the aperture 1214. The apertures 1214, 1314, and 1414 are all directly adjacent to the first long side 102 of the transducer array 100, and thus the centroid 1216 of the aperture 1214 is closer to the first long side 102 of the transducer array 100 than the centroid 1316 of the aperture 1314 is, and the centroid 1316 of the aperture 1314 is closer to the first long side 102 of the transducer array 100 than the centroid 1416 of the aperture 1414 is. It should be appreciated that any of the apertures illustrated or described herein may vary in elevational width as a function of imaging depth in the manner illustrated in FIGS. 12-14.

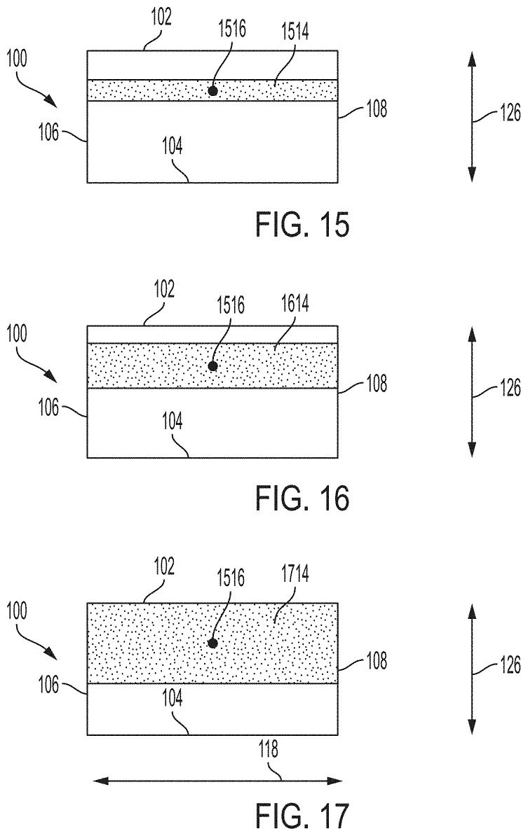

FIGS. 15-17 schematically illustrate more examples of apertures that vary as a function of imaging depth, in accordance with certain embodiments described herein. FIG. 15 illustrates an aperture 1514 used for a first imaging depth, FIG. 16 illustrates an aperture 1614 used for a second imaging depth that is deeper than the first imaging depth, and FIG. 17 illustrates an aperture 1714 used for a third imaging depth that is deeper than the second imaging depth. The aperture 1714 is wider along the elevational dimension 126 than the aperture 1614, and the aperture 1614 is wider along the elevational dimension 126 than the aperture 1514. The aperture 1514, the aperture 1614, and the aperture 1714 all have the same centroid 1516. It should be appreciated that any of the apertures illustrated or described herein may vary in elevational width as a function of imaging depth in the manner illustrated in FIGS. 15-17.

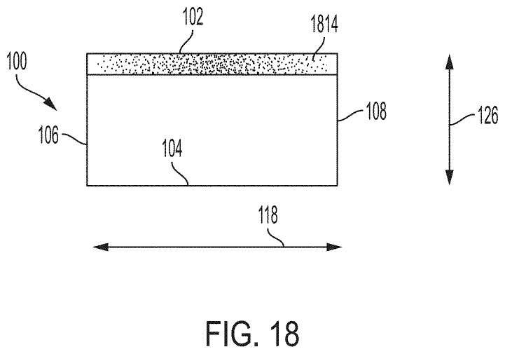

FIG. 18 schematically illustrates another example aperture 1814, in accordance with certain embodiments described herein. As illustrated, the aperture 1814 varies along the azimuthal dimension 118. In particular, the signals received by the aperture 1814 are averaged with a weighted sum along the azimuthal dimension 118, such that the signals received nearer to the center of the azimuthal dimension 118 are weighted more than signals received farther from the center of the azimuthal dimension 118. It should be appreciated that any of the apertures illustrated or described herein may vary across the azimuthal dimension 118 in the manner illustrated in FIG. 18. It should also be appreciated that any of the apertures illustrated herein may be used for transmitting or receiving ultrasound waves, or both.

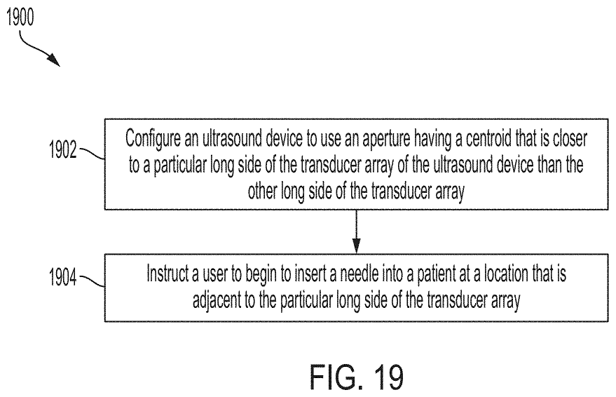

FIGS. 19-23 illustrate example processes 1900, 2000, 2100, 2200, and 2300 in accordance with certain embodiments described herein. The processes 1900, 2000, 2100, 2200, and 2300 are performed by a processing device in an ultrasound system. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself may perform these processes.

Turning to FIG. 19, in act 1902 of the process 1900, the processing device configures the ultrasound device to use an aperture having a centroid that is closer to a particular long side of the transducer array than the other long side of the transducer array. For example, the processing device may configure the ultrasound device with any of the apertures illustrated in FIGS. 3-18, or any of the variations or alternatives described. The process 1900 proceeds from act 1902 to act 1904.

In act 1904, the processing device instructs a user to begin to insert a needle adjacent into a patient at a location that is to the particular long side of the transducer array that is closer to the aperture. For example, the processing device may display such an instruction. In some embodiments, the instruction to insert the needle adjacent into the patient at the location that is to the particular long side of the transducer array may be an instruction to insert the needle into the patient at a location that is adjacent to a particular face of the ultrasound device, where the particular face of the ultrasound device is adjacent to the particular long side of the transducer array. In some embodiments, the ultrasound device may have a marking on one of its faces that is adjacent to the particular long side of the transducer array, but not have the same marking on the other face of the ultrasound device. The processing device may instruct the user to begin to insert the needle into the patient at a location that is adjacent to the face of the ultrasound device that has the marking, or instruct the user to begin to insert the needle into the patient at a location that is adjacent to the face of the ultrasound device that does not have the marking.

In some embodiments, a camera on the processing device may capture an image or video of the ultrasound device. For example, the user may hold the ultrasound device on the subject with one hand and hold the processing device with the other hand such that the ultrasound device is in view of the camera on the processing device. The processing device may display an augmented reality interface depicting the image or video of the ultrasound device with an indication of the particular face of the ultrasound device superimposed on the image or video. For example, the indication may be an arrow that points to the particular face of the ultrasound device as depicted in the image or video. In some embodiments, a statistical model may be trained to determine where in the image or video to display the indication. The statistical model may be trained on multiple images or videos depicting the ultrasound device, each of the images or videos labeled with an indication of the particular face of the ultrasound device. In some embodiments, the processing device may use a fiducial marker (e.g., a marking on the ultrasound device) to determine where in the image or video to display the indication. In response to the instruction to begin to insert the needle into the patient at a location that is adjacent to the particular long side of the transducer array, the user may begin to insert the needle into the patient at a location that is adjacent to the particular long side of the transducer array.

In some embodiments, act 1904 may be absent. For example, a user may already know to begin to insert the needle into the patient at a location that is adjacent to the particular long side of the transducer array. In some embodiments, act 1902 may be absent. For example, the ultrasound device may already be configured to use an aperture having a centroid that is closer to a particular long side of the transducer array than the other long side of the transducer array.

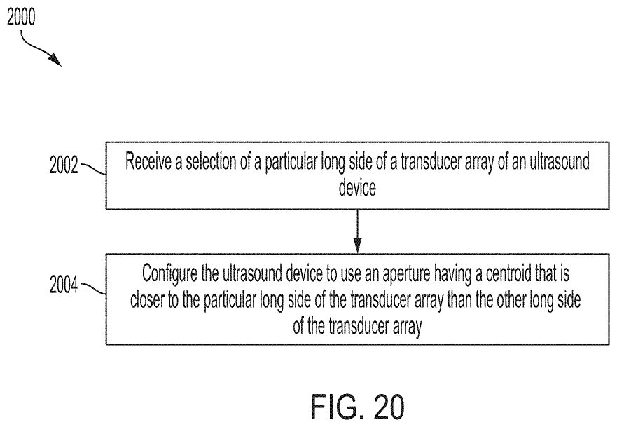

Turning to FIG. 20, in act 2002 of the process 2000, the processing device receives a selection of a particular long side of the transducer array. In some embodiments, the processing device may display two options, each corresponding to one of the long sides of the transducer array. In some embodiments, the processing device may also display an indication of one of the long sides of the transducer array, namely the side that is closer to the aperture. For example, the option corresponding to the long side of the transducer array that is closer to the aperture may be highlighted. Prior to any selections by the user, the aperture may be closer to a default long side of the transducer array. The processing device may receive a selection of one of the two options. As one example of use, a user may decide that the needle will begin to be inserted into the patient at a location that is adjacent to a particular long side of the transducer array. If the processing device indicates that the aperture is already closer to that particular long side of the transducer array rather than the other long side (e.g., the corresponding option is highlighted), the user may not need to select that option. However, if the processing device indicates that the aperture is not already closer to that particular long side of the transducer array (e.g., the corresponding option is not highlighted), the user may select that long side of the transducer array by selecting the corresponding option from the two options. In some embodiments, the processing device may not display an indication of one of the long sides of the transducer array, namely the side that is closer to the aperture, and the user may select the option corresponding to the particular long side of the transducer array regardless of whether the aperture is already closer to the particular long side of the transducer array.

In some embodiments, the processing device may display one option. Prior to any selections by the user, the aperture may be closer to a default long side of the transducer array. Every time the processing device receives a selection of the option, the long side of the transducer array that was not previously selected may be selected. Thus, by selecting the option on the processing device, the user may switch from selecting one side of the transducer array to selecting the other side of the transducer array. As one example of use, a user may begin to insert a needle into the patient at a location that is adjacent to a particular long side of the transducer array. The user may view one or more ultrasound images collected by the ultrasound device, and if the one or more ultrasound images do not depict the needle with acceptable brightness, this may be an indication that the aperture is closer to the other long side of the transducer array. The user may therefore select the option to switch which long side of the transducer array is closer to the aperture. The process 2000 proceeds from act 2002 to act 2004.

In act 2004, the processing device configures the ultrasound device to use an aperture having a centroid that is closer to the particular long side of the transducer array selected in act 2002 than the other long side of the transducer array. For example, the processing device may configure the ultrasound device with any of the apertures illustrated in FIGS. 3-18, or any of the variations or alternatives described.

In some embodiments, act 2002 may be absent. For example, a particular long side of the transducer array may already be selected by default. In some embodiments, act 2004 may be absent. For example, the ultrasound device may already be configured to use an aperture having a centroid that is closer to a particular long side of the transducer array than the other long side of the transducer array.

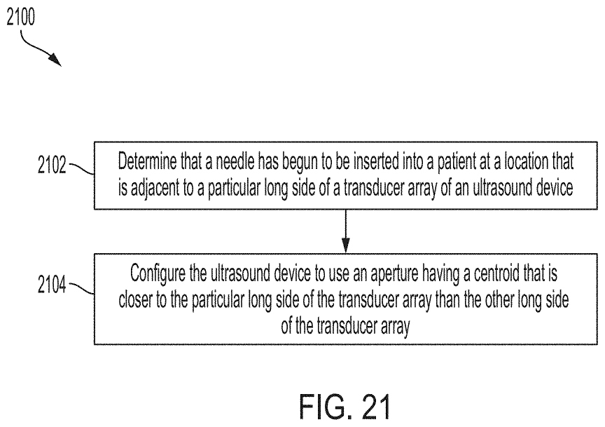

Turning to FIG. 21, in act 2102 of the process 2100, the processing device determines that a needle has begun to be inserted into a patient at a location that is adjacent to a particular long side of the transducer array. In some embodiments, the processing device may use one or more statistical models trained to determine from one or more ultrasound images that a needle has begun to be inserted into the patient at a location that is adjacent to a particular long side of the transducer array. The statistical models may be trained on multiple ultrasound images, each labeled with whether a needle has been inserted adjacent to the long side of the transducer array that is closer to the aperture or whether the needle has been inserted into the patient at a location that is adjacent to the long side of the transducer array that is farther from the aperture. Thus, the processing device may input one or more ultrasound images to the statistical models, and the statistical models may determine whether the needle has been inserted into the patient at a location that is adjacent to the long side of the transducer array that is closer to the aperture or whether the needle has been inserted into the patient at a location that is adjacent to the long side of the transducer array that is farther from the aperture. Based on which particular long side of the transducer array is currently closer to the aperture, the processing device may determine which particular long side of the transducer array is adjacent to the needle.

In some embodiments, the processing device may use one or more statistical models trained to determine the level of brightness of a needle visible in one or more ultrasound images. The statistical models may be trained on multiple ultrasound images, each labeled with a level of brightness of a needle visible in the ultrasound image. Thus, the processing device may input one or more ultrasound images to the statistical models, and the statistical models may determine the level of brightness of a needle visible in the one or more ultrasound images. If the statistical models determine that the level of brightness of the needle exceeds a threshold brightness level, the processing device may determine that the needle has been inserted into the patient at a location that is adjacent to the long side of the transducer array that is closer to the aperture. If the statistical models determine that the level of brightness of the needle does not exceed a threshold brightness level, the processing device may determine that the needle has been inserted into the patient at a location that is adjacent to the long side of the transducer array that is not closer to the aperture. The process 2100 proceeds from act 2102 to act 2104.

In act 2104, the processing device configures the ultrasound device to use an aperture having a centroid that is closer to the particular long side of the transducer array determined in act 2102. For example, the processing device may configure the ultrasound device with any of the apertures illustrated in FIGS. 3-18, or any of the variations or alternatives described.

In some embodiments, act 2104 may be absent. For example, the ultrasound device may already be configured to use an aperture having a centroid that is closer to the particular long side of the transducer array than the other long side of the transducer array.

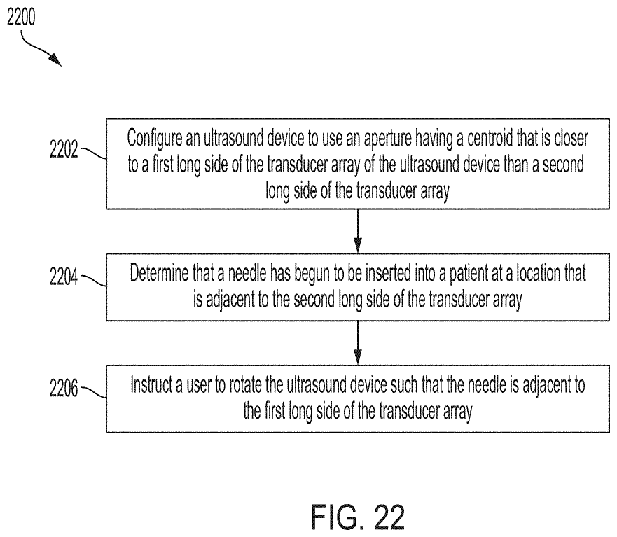

Turning to FIG. 22, in act 2202 of the process 2200, the processing device configures the ultrasound device to use an aperture having a centroid that is closer to a first side of the transducer array than the second side of the transducer array. Further description of configuring the ultrasound device to use an aperture having a centroid that is closer to a particular side of a transducer array may be found with reference to act 1902. The process 2200 proceeds from act 2202 to act 2204.

In act 2204, the processing device determines that a needle has begun to be inserted into a patient at a location that is adjacent to the second long side of the transducer array. Further description of determining that the needle has begun to be inserted into a patient at a location that is adjacent to a particular long side of the transducer array may be found with reference to act 2102. The process 2200 proceeds from act 2204 to act 2206.

In act 2206, the processing device instructs a user to rotate the ultrasound device such that the needle is adjacent to the first long side of the transducer array. In some embodiments, the processing device may instruct the user to rotate the ultrasound device 180 degrees about its longitudinal axis. In some embodiments, the ultrasound device may have a marking on its face that is adjacent to the first long side of the transducer array, but not have the same marking on the other face of the ultrasound device that is adjacent to the second long side of the transducer array, and the processing device may instruct the user to rotate the ultrasound device such that the face of the ultrasound device that has the marking is adjacent to the needle. In some embodiments, the ultrasound device may have a marking on its face that is adjacent to the second long side of the transducer array, but not have the same marking on the other face of the ultrasound device that is adjacent to the first long side of the transducer array, and the processing device may instruct the user to rotate the ultrasound device such that the face of the ultrasound device that does not have the marking is adjacent to the needle.

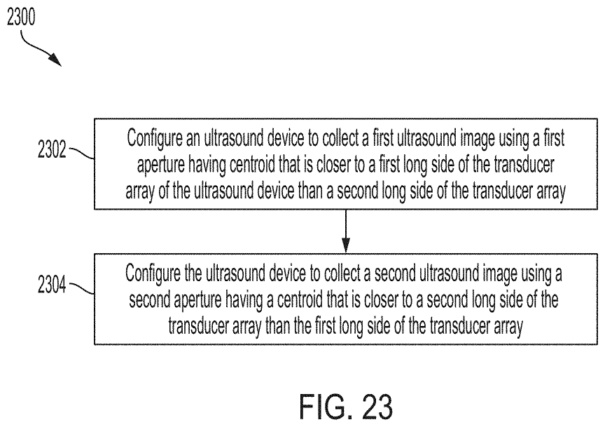

Turning to FIG. 23, in act 2302 of the process 2300, the processing device configures the ultrasound device to collect a first ultrasound image using a first aperture having a centroid that is closer to a first long side of the transducer array than a second long side of the transducer array. For example, the first aperture may be the aperture 314. The process 2300 proceeds from act 2302 to act 2304.

In act 2304, the processing device configures the ultrasound device to collect a second ultrasound image using a second aperture having a centroid that is closer to the second long side of the transducer array than the first long side the transducer array. For example, the second aperture may be the aperture 1114.

In some embodiments, the processing device may alternate between acts 2302 and act 2304. In other words, the processing device may alternate between collecting an ultrasound image using the first aperture and collecting an ultrasound image using the second aperture. For example, the processing device may configure the ultrasound device to collect ultrasound images at a frame rate FR, where the ultrasound device collects ultrasound images using the first aperture at a frame rate FR/2 and the ultrasound device collects ultrasound images using the second aperture at a frame rate FR/2.

In some embodiments, the processing device may display both the first ultrasound image and the second ultrasound image, and the processing device may receive a selection of either the first ultrasound image or the second ultrasound image. For example, in one of the images, a needle may visible with a higher level of brightness than in the other image. The user may select the image in which the needle is visible with the higher level of brightness, and the processing device may configure the ultrasound device to continue to collect ultrasound image just using the aperture with which the selected image was collected.

In some embodiments, the processing device may use one or more statistical models trained to determine the level of brightness of a needle visible in an ultrasound image. The statistical models may be trained on multiple ultrasound images, each labeled with a level of brightness of a needle visible in the ultrasound image. Thus, the processing device may input the first and second ultrasound images to the statistical models, and the statistical models may determine the level of brightness of the needle visible in the first or second ultrasound image. The processing device may then configure the ultrasound device to continue to collect ultrasound image just using the aperture with which the image depicting the needle with a higher brightness level was collected.

In some embodiments, the processing device may use one or more statistical models trained to determine from an ultrasound image whether a needle has begun to be inserted into the patient at a location that is adjacent to a long side of the transducer array that is closer to the aperture or father than the aperture. The statistical models may be trained on multiple ultrasound images, each labeled with whether a needle has been inserted adjacent into the patient at a location that is to the long side of the transducer array that is closer to the aperture or whether the needle has been inserted into the patient at a location that is adjacent to the long side of the transducer array that is farther from the aperture. Thus, the processing device may input the first and second ultrasound images to the statistical models, and the statistical models may determine whether the first or second ultrasound image was collected with an aperture closer to a long side of the transducer array that is adjacent to the needle. The processing device may then configure the ultrasound device to continue to collect ultrasound images just using the aperture that is closer to the long side of the transducer array adjacent to the needle.

It should be appreciated that the first aperture may be any of the apertures illustrated in FIGS. 3-18, or any of the variations or alternatives described. The second aperture may also be any of the apertures illustrated in FIGS. 3-18, or any of the variations or alternatives described, as long as the second aperture is adjacent to the opposite long side of the transducer array than the first aperture.

In some embodiments, the processing device may configure the ultrasound device to collect an ultrasound image using an aperture covering the whole transducer array, determine based on the ultrasound image that the needle is being adjacent to a particular long side of the transducer array (e.g., using any of the methods described with reference to FIG. 23), and configure the ultrasound device to use an aperture that is adjacent to that particular long side of the transducer array. In such embodiments, the processing device may be capable of generating an ultrasound image from the whole transducer array, without requiring any averaging.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps

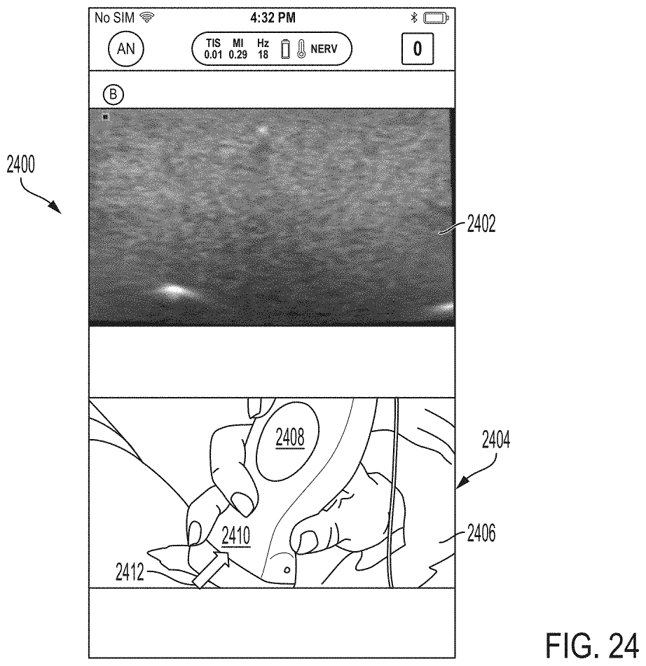

FIGS. 24-28 illustrate example graphical user interfaces (GUIs) 2400, 2500, 2600, 2700, and 2800 in accordance with certain embodiments described herein. The GUIs 2400, 2500, 2600, 2700, and 2800 are displayed by a processing device in an ultrasound system. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself may display the GUIs.

Turning to FIG. 24, the GUI 2400 includes an ultrasound image 2402 and an augmented reality (AR) interface 2404. The AR interface 2404 includes a video 2406 and an arrow 2412. The video 2406 depicts an ultrasound device 2408 that includes a face 2410 adjacent to a particular long side of a transducer array. The arrow 2412 points to the face 2410 adjacent to the particular long side of the transducer array.

As described with reference to FIG. 19, the processing device may have configured the ultrasound device 2408 to use an aperture having a centroid that is closer to one long side of the transducer array than the other long side of the transducer array. The arrow 2412 may constitute an instruction to begin to insert a needle into a patient at a location that is adjacent to the face 2410 of the ultrasound device 2408 that is adjacent to that long side of the transducer array. A camera on the processing device may capture the video 2406. For example, the user may hold the ultrasound device 2408 on the subject with one hand and hold the processing device with the other hand such that the ultrasound device 2408 is in view of the camera on the processing device. In some embodiments, the processing device may use a fiducial marker (e.g., a marking on the ultrasound device 2408) to determine where in the video 2406 to display the arrow 2412. In some embodiments, a statistical model may be trained to determine where in the video 2406 to display the arrow 2412. The statistical model may be trained on multiple images or videos depicting an ultrasound device, each of the images or videos labeled with an indication of the face of the ultrasound device that is adjacent to a particular long side of the ultrasound device. In response to the arrow 2412, the user may begin to insert the needle into the patient at a location that is adjacent to the long side of the transducer array that is closer to the aperture.

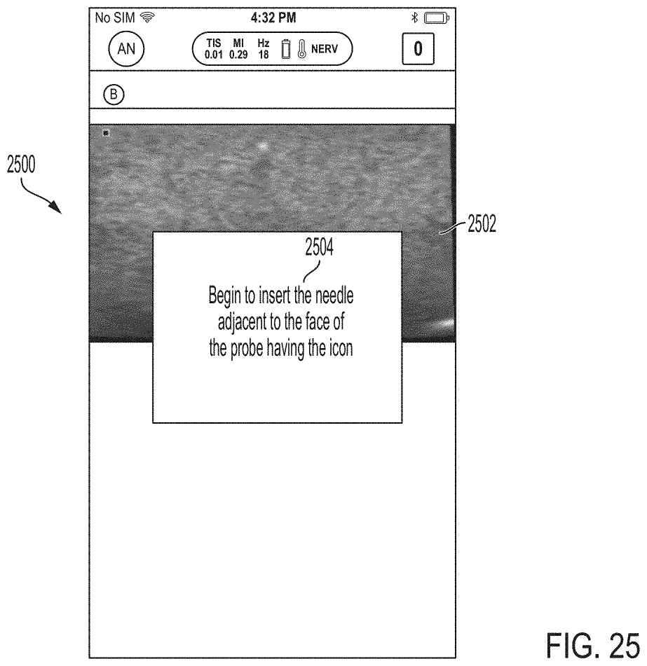

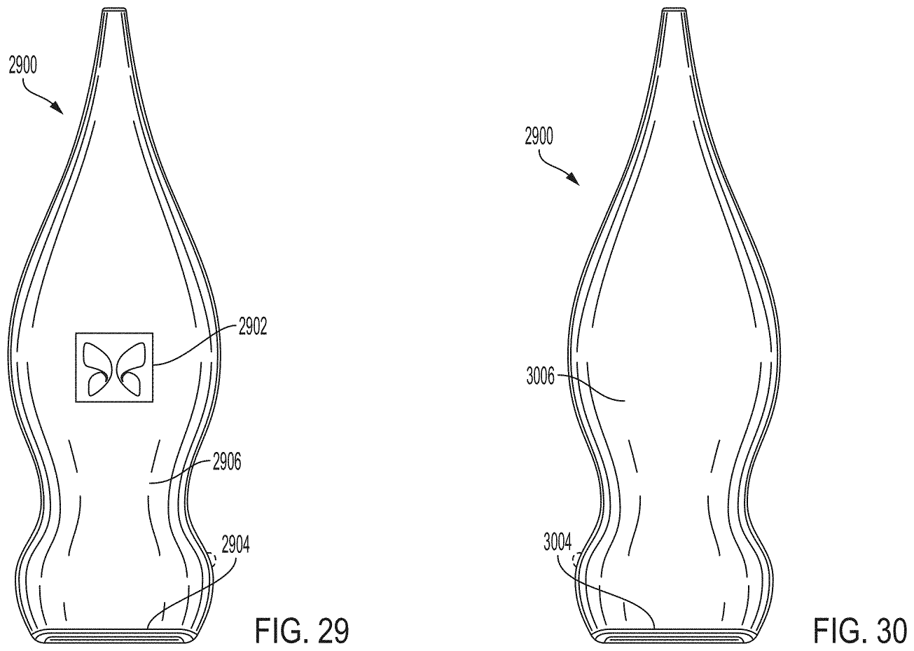

Turning to FIG. 25, the GUI 2500 includes an ultrasound image 2502 and text 2504. The text 2504 instructs the user to begin to insert a needle into the patient at a location that is adjacent to a face of an ultrasound device that has an icon. As described with reference to FIG. 19, the processing device may have configured the ultrasound device to use an aperture having a centroid that is closer to a long side of the transducer array than the other long side of the transducer array. Furthermore, in some embodiments (and as illustrated in FIGS. 29-30), the ultrasound device may have an icon on the face of the ultrasound device that is closer to this long side of the transducer array, but not have the same icon on the other long side of the ultrasound device. The text 2504 thus instructs the user to begin to insert the needle into the patient at a location that is adjacent to this face of the ultrasound device, or equivalently, adjacent to this long side of the transducer array. If the processing device configured the ultrasound device to use an aperture having a centroid that is closer to the other side of the transducer array, the text 2504 may instruct the user to begin to insert the needle into the patient at a location that is adjacent to the face of the ultrasound device that does not have the icon.

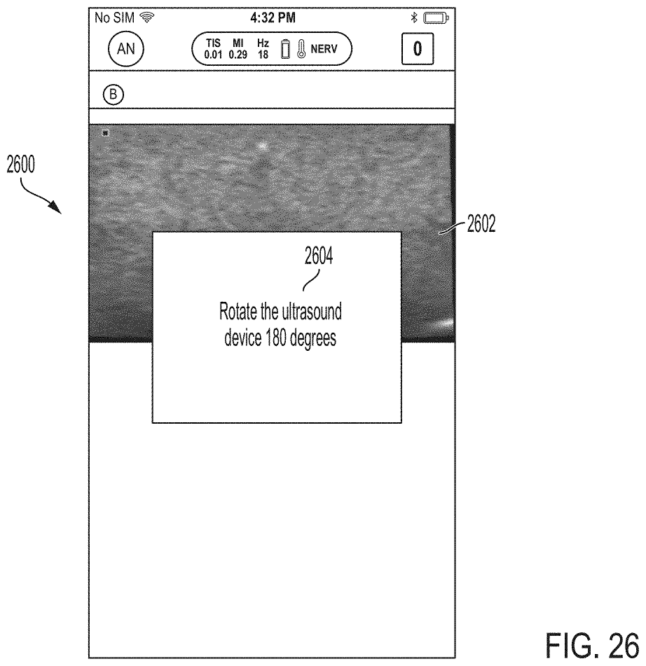

Turning to FIG. 26, the GUI 2600 includes an ultrasound image 2602 and text 2604. The text 2604 instructs the user to rotate the ultrasound device 180 degrees. As described with reference to FIG. 22, in some embodiments the processing device may configure the ultrasound device to use an aperture having a centroid that is closer to a first side of the transducer array than the second side of the transducer array and then determine that a needle has begun to be inserted into a patient at a location that is adjacent to the second long side of the transducer array. The text 2604 thus instructs the user to rotate the ultrasound device such that the needle is adjacent to the first long side of the transducer array. In some embodiments, the processing device may display text instructing the user to rotate the ultrasound device 180 degrees about its longitudinal axis. In some embodiments, the ultrasound device (as illustrated in FIGS. 29-30) may have a marking on its face that is adjacent to the first long side of the transducer array, but not have the same marking on the other face of the ultrasound device that is adjacent to the second long side of the transducer array, and the processing device may display text instructing the user to rotate the ultrasound device such that the face of the ultrasound device that has the marking is adjacent to the needle. In some embodiments, the ultrasound device (as illustrated in FIGS. 29-30) may have a marking on its face that is adjacent to the second long side of the transducer array, but not have the same marking on the other face of the ultrasound device that is adjacent to the first long side of the transducer array, and the processing device may display text instructing the user to rotate the ultrasound device such that the face of the ultrasound device that does not have the marking is adjacent to the needle.

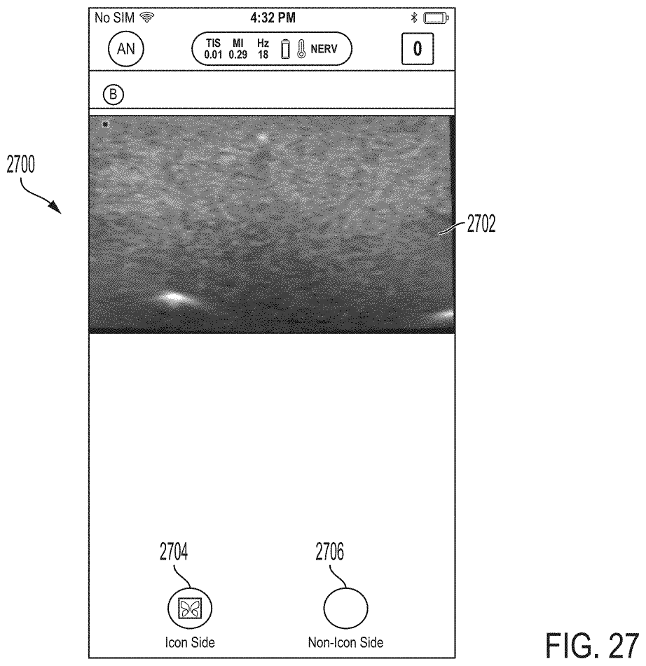

Turning to FIG. 27, the GUI 2700 includes an ultrasound image 2702, a first option 2704, and a second option 2706. The first option 2704 corresponds to one long side of the transducer array of the ultrasound device, and the second option 2706 corresponds to the other long side of the transducer array of the ultrasound device. In FIG. 27, the first option 2704 may correspond to the long side of the transducer array adjacent to the face of the ultrasound device that has an icon (as illustrated in FIG. 29). The second option 2706 may correspond to the long side of the transducer array adjacent to the face of the ultrasound device that does not have the icon (as illustrated in FIG. 30). The first option 2704 is highlighted while the second option 2706 is not highlighted. This may indicate that the ultrasound device is configured such that the aperture is closer to the long side of the transducer array corresponding to the first option 2704. As described with reference to FIG. 20, if a user decides that the needle will begin to be inserted into the patient at a location that is adjacent to the long side of the transducer array corresponding to the first option 2704, the user may not need to select the first option 2704, as the ultrasound device is already configured such that the aperture is closer to the long side of the transducer array corresponding to the first option 2704. However, if the user decides that the needle will begin to be inserted into the patient at a location that is adjacent to the long side of the transducer array corresponding to the second option 2706, the user may select the second option 2706. In response to receiving the selection of the second option 2706, the processing device may highlight the second option 2706, remove the highlighting of the first option 2704, and configure the ultrasound device such that the aperture is closer to the long side of the transducer array corresponding to the second option 2706.

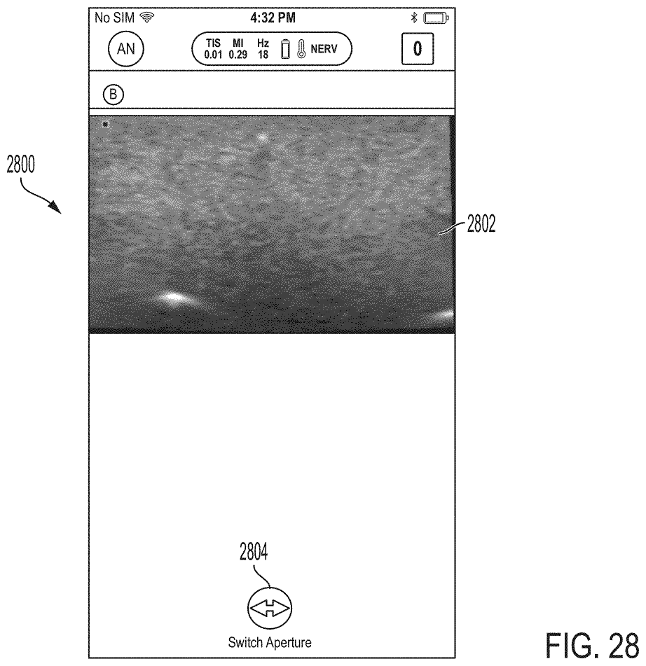

Turning to FIG. 28, the GUI 2800 includes an ultrasound image 2802 and an option 2804. The ultrasound device may be configured such that the aperture is closer to a default long side of the transducer array. Every time the processing device receives a selection of the option 2804, the long side of the transducer array that was not previously selected may be selected. Thus, by selecting the option 2804, the user may switch from selecting one side of the transducer array to the other side of the transducer array. A user may begin to insert a needle into a patient at a location that is adjacent to a particular long side of the transducer array. The user may view one or more ultrasound images collected by the ultrasound device, and if the one or more ultrasound images do not depict the needle with acceptable brightness, this may be an indication that the aperture is closer to the other long side of the transducer array. The user may therefore select the option 2804. In response to receiving the selection of the option 2804, the processing device may configure the ultrasound device such that the aperture is closer to the selected long side of the transducer array.

FIGS. 29 and 30 illustrate an ultrasound device 2900, in accordance with certain embodiments described herein. FIG. 29 is a view of one face 2906 the ultrasound device 2900. FIG. 30 is a view of another face 3006 of the ultrasound device 2900. The face 2906 of the ultrasound device 2900 includes an icon 2902 and is adjacent to a long side 2904 of the transducer array of the ultrasound device 2900. The face 3006 of the ultrasound device 2900 does not include the icon 2902 and is adjacent to a long side 3004 of the transducer array of the ultrasound device 2900. Thus, as described with reference to FIGS. 19 and 25, a processing device may instruct a user to begin to insert a needle into a patient at a location that is adjacent to the long side 2904 of the transducer array by instructing the user to begin to insert the needle into the patient at a location that is adjacent to the face 2906 of the ultrasound device 2900 that has the icon 2902. The processing device may instruct the user to begin to insert the needle into the patient at a location that is adjacent to the long side 3004 of the transducer array by instructing the user to begin to insert the needle into the patient at a location that is adjacent to the face 3006 of the ultrasound device 2900 that does not have the icon 2902. It should be noted that in some embodiments, the face 3006 of the ultrasound device 2900 may have other icons, but not the icon 2902. The form of the icon 2902 is not limiting.

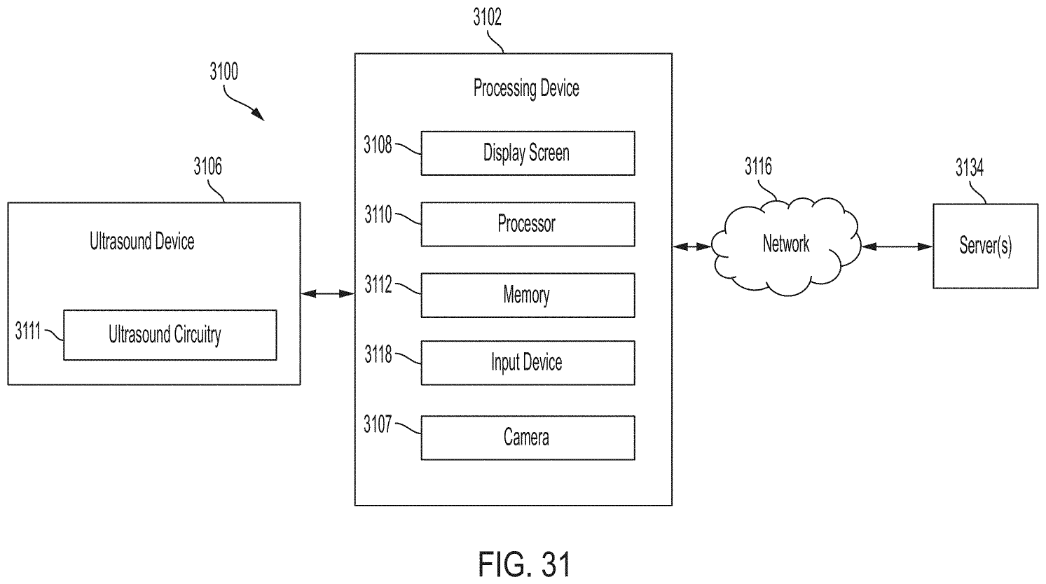

FIG. 31 illustrates a schematic block diagram of an example ultrasound system 3100 upon which various aspects of the technology described herein may be practiced. The ultrasound system 3100 includes an ultrasound device 3106, a processing device 3102, a network 3116, and one or more servers 3134.

The ultrasound device 3106 includes ultrasound circuitry 3111. The processing device 3102 includes a camera 3107, a display screen 3108, a processor 3110, a memory 3112, and an input device 3118. The processing device 3102 is in wired (e.g., through a lightning connector or a mini-USB connector) and/or wireless communication (e.g., using BLUETOOTH, ZIGBEE, and/or WiFi wireless protocols) with the ultrasound device 3106. The processing device 3102 is in wireless communication with the one or more servers 3134 over the network 3116. However, the wireless communication with the processing device 3134 is optional.

The ultrasound device 3106 may be configured to generate ultrasound data that may be employed to generate an ultrasound image. The ultrasound device 3106 may be constructed in any of a variety of ways. In some embodiments, the ultrasound device 3106 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 3111 may be configured to generate the ultrasound data. The ultrasound circuitry 3111 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 3111 (e.g., transmit circuitry, receive circuitry, control circuitry, power management circuitry, and processing circuitry) to form a monolithic ultrasound device. The ultrasound device 3106 may transmit ultrasound data and/or ultrasound images to the processing device 3102 over a wired (e.g., through a lightning connector or a mini-USB connector) and/or wireless (e.g., using BLUETOOTH, ZIGBEE, and/or WiFi wireless protocols) communication link.

Referring now to the processing device 3102, the processor 3110 may include specially-programmed and/or special-purpose hardware such as an application-specific integrated circuit (ASIC). For example, the processor 3110 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network. The processing device 3102 may be configured to process the ultrasound data received from the ultrasound device 3106 to generate ultrasound images for display on the display screen 3108. The processing may be performed by, for example, the processor 3110. The processor 3110 may also be adapted to control the acquisition of ultrasound data with the ultrasound device 3106. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing device 3102 may be configured to perform certain of the processes described herein using the processor 3110 (e.g., one or more computer hardware processors) and one or more articles of manufacture that include non-transitory computer-readable storage media such as the memory 3112. The processor 3110 may control writing data to and reading data from the memory 3112 in any suitable manner. To perform certain of the processes described herein, the processor 3110 may execute one or more processor-executable instructions stored in one or more non-transitory computer-readable storage media (e.g., the memory 3112), which may serve as non-transitory computer-readable storage media storing processor-executable instructions for execution by the processor 3110. The camera 3107 may be configured to detect light (e.g., visible light) to form an image. The camera 3107 may be on the same face of the processing device 3102 as the display screen 3108. The display screen 3108 may be configured to display images and/or videos, and may be, for example, a liquid crystal display (LCD), a plasma display, and/or an organic light emitting diode (OLED) display on the processing device 3102. The input device 3118 may include one or more devices capable of receiving input from a user and transmitting the input to the processor 3110. For example, the input device 3118 may include a keyboard, a mouse, a microphone, touch-enabled sensors on the display screen 3108, and/or a microphone. The display screen 3108, the input device 3118, and the camera 3107 may be communicatively coupled to the processor 3110 and/or under the control of the processor 3110.

It should be appreciated that the processing device 3102 may be implemented in any of a variety of ways. For example, the processing device 3102 may be implemented as a handheld device such as a mobile smartphone or a tablet. Thereby, a user of the ultrasound device 3106 may be able to operate the ultrasound device 3106 with one hand and hold the processing device 3102 with another hand. In other examples, the processing device 3102 may be implemented as a portable device that is not a handheld device, such as a laptop. In yet other examples, the processing device 3102 may be implemented as a stationary device such as a desktop computer.

The processing device 3102 may be connected to the network 3116 over a wired connection (e.g., via an Ethernet cable) and/or a wireless connection (e.g., over a WiFi network). The processing device 3108 may thereby communicate with (e.g., transmit data to or receive data from) the one or more servers 3134 over the network 3116. For example, a party may provide from the one or more servers 3134 to the processing device 3108 processor-executable instructions for storing in one or more non-transitory computer-readable storage media (e.g., the memory 3112) which, when executed, may cause the processing device 3108 to perform certain of the processes described herein. For further description of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 and published as U.S. Pat. App. Publication No. 2017-0360397 A1 (and assigned to the assignee of the instant application).

FIG. 31 should be understood to be non-limiting. For example, the ultrasound system 3100 may include fewer or more components than shown, the processing device 3102 may include fewer or more components than shown, and the ultrasound device 3106 may include fewer or more components than shown.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising:
an ultrasound device having a transducer array, the ultrasound device configured to use an aperture having a centroid that is closer to a first long side of the transducer array than a second long side of the transducer array.

2. The apparatus of claim 1, wherein the aperture is rectangular.

3. The apparatus of claim 1, wherein the aperture is between or equal to approximately 1-10 mm along an elevational dimension of the transducer array.

4. The apparatus of claim 1, wherein the aperture covers between or equal to approximately 20-30% of an elevational dimension of the transducer array.

5. The apparatus of claim 1, wherein the aperture is not centered along an elevational dimension of the transducer array.

6. The apparatus of claim 1, wherein the aperture is directly adjacent to the first long side of the transducer array.

7. The apparatus of claim 1, wherein the aperture is centered with respect to an azimuthal dimension of the transducer array.

8. The apparatus of claim 1, wherein the aperture extends along an entire length of an azimuthal dimension of the transducer array.

9. The apparatus of claim 1, wherein the aperture does not extend along an entire length of an azimuthal dimension of the transducer array.

10. The apparatus of claim 1, wherein the aperture is not centered with respect to an azimuthal dimension of the transducer array.

11. The apparatus of claim 1, wherein the aperture is directly adjacent to a first short side of the transducer array.

12. The apparatus of claim 1, wherein the aperture is not directly adjacent to the first long side of the transducer array.

13. The apparatus of claim 1, wherein the aperture is not rectangular.

14. The apparatus of claim 1, wherein a width of the aperture along an elevational dimension of the transducer array varies with imaging depth.

15. The apparatus of claim 1, wherein the ultrasound device is further configured to average, with a weighted sum along an azimuthal dimension of the transducer array, signals received by the aperture.

16. An apparatus, comprising:
a processing device in operative communication with an ultrasound device having a transducer array, the processing device configured to instruct a user to begin to insert an object into a patient at a location that is adjacent to a particular long side of the transducer array.

17. The apparatus of claim 16, wherein the processing device is configured, when instructing the user to begin to insert the object into the patient at the location that is adjacent to the particular long side of the transducer array, to instruct the user to insert the object into the patient at a location that is adjacent to a particular face of the ultrasound device.

18. The apparatus of claim 17, wherein the particular face of the ultrasound device is adjacent to the particular long side of the transducer array.

19. The apparatus of claim 17, wherein the processing device is configured, when instructing the user to insert the object into the patient at the location that is adjacent to the particular face of the ultrasound device, to instruct the user to insert the object into the patient at a location that is adjacent to a particular face of the ultrasound device having a particular marking.

20. The apparatus of claim 17, wherein the processing device is configured, when instructing the user to insert the object into the patient at the location that is adjacent to the particular face of the ultrasound device, to instruct the user to insert the object into the patient at a location that is adjacent to a particular face of the ultrasound device lacking a particular marking.

21. The apparatus of claim 16, wherein the processing device is further configured to configure the ultrasound device to use an aperture having a centroid that is closer to the particular long side of the transducer array than another long side of the transducer array.

22. The apparatus of claim 16, wherein the object is a needle.

23. An apparatus, comprising:
a processing device in operative communication with an ultrasound device having a transducer array, the processing device configured to receive a selection of a particular long side of the transducer array.

24. The apparatus of claim 23, wherein:
the processing device is further configured to display two options, each corresponding to a particular long side of the transducer array; and
the processing device is configured, when receiving the selection of the particular long side of the transducer array, to receive a selection of one of the two options.

25. The apparatus of claim 23, wherein the processing device is further configured to configure the ultrasound device to use an aperture having a centroid that is closer to the particular long side of the transducer array than another long side of the transducer array.

26. The apparatus of claim 25, wherein the processing device is further configured to display an indication of the particular long side of the transducer array that is closer to the aperture by highlighting one of the two options that corresponds to the particular long side of the transducer array.

27. The apparatus of claim 23, wherein:
the processing device is further configured to display an option; and
the processing device is configured, when receiving the selection of the particular long side of the transducer array, to receive a selection of the option.

28. The apparatus of claim 27, wherein the processing device is configured, based on receiving the selection of the option, to configure the ultrasound device to switch from using an aperture having a centroid that is closer to a first long side of the transducer array to using an aperture having a centroid that is closer to a second long side of the transducer array.
